(19) United States
(12) Patent Application Publication
(10) Pub. No.: US 2016/0361557 A1
(43) Pub. Date: Dec. 15, 2016

(54) APPARATUS AND METHOD FOR REPAIRING AND REGENERATING CARDIAC TISSUES AND FOR THE ELECTRO-PHYSIOLOGICAL, METABOLIC OPTIMIZATION OF THE HEART

(71) Applicants: Salvatore RINALDI, Scandicci (IT); Vania FONTANI, Scandicci (IT)

(72) Inventors: Salvatore RINALDI, Scandicci (IT); Vania FONTANI, Scandicci (IT)

(21) Appl. No.: 15/115,190

(22) PCT Filed: Jan. 31, 2014
(86) PCT No.: PCT/IB2014/058701

§ 371 Date: Jul. 28, 2016

(51) Int. Cl. A61N 1/40; A61N 1/08; A61M 37/00; A61N 5/04

(52) U.S. Cl. CPC A61N 1/40; A61N 5/045; A61N 1/08; A61M 37/00

(57) ABSTRACT

An apparatus comprising a catheter (10), associated with a radiofrequency radio-electric signal generator, adapted to convey said radio-electric signal to the cardiac surfaces in order to repair, regenerate and normalize the electro-physiological, metabolic activities of the heart. The generator comprises a transmitter (13) adapted to produce a radiofrequency electromagnetic field, an antenna (14) to emit said radiofrequency electromagnetic field, a control module (18) and a user interface (19). A conductor (15) connects at one end to the reference ground of the transmitter circuitry and at the other end to the electrode (12).

### FIELD OF THE INVENTION

The present invention relates to the technical field of methods and apparatuses for treating heart diseases. In particular, the present invention relates to methods and apparatuses for improving cardiac function using electro-stimulation based therapies.

### PRIOR ART

In the prior art, there are two types of apparatuses used for cardiac therapeutic treatments comprising catheters connected to electric signal generators.

A first type comprises catheters connected to electric stimulators adapted either to cure or to prevent some types of cardiac arrhythmias. These catheters are used to electrically simulate the autonomous nerves which innervate the heart. Devices of this type generally comprise an endovascular electrode adapted to be implanted in the patient and configured to provide such a stimulation when an imbalance is foreseen or registered. Apparatuses of this type make use of electric signals with a frequency in the range from a few Hertz to a few tens of Hertz.

A second type comprises catheters connected to radiofrequency (RF) electric signal generators. Apparatuses of this type are adapted to locally give energy, in order to destroy diseased tissues by means of a procedure, named ablation. The radiofrequency energy substantially causes a local overheating which destroys the selected cells and tissues.

In cardiology, apparatuses of this type are widely used for ablating diseased tissues and abnormal electric conduction nodes and/or pathways. The catheters of these apparatuses are not implanted, but are instead adapted to be handled by the operator who places them appropriately at the concerned region and then carries out the ablation by irradiating said region with a radiofrequency electric signal emitted by the catheter itself. Therefore, the handle of the catheter of the apparatuses of this type is usually mechanically connected to the distal part by means of appropriate tie-rods in order to place it and/or bend it correctly. Apparatuses of this type make use of electric signals with a frequency from 500 kHz to 2.5 GHz with powers capable of creating a local overheating sufficient to destroy the selected tissues. In general, the electric powers used range from a minimum of a few tens of Watts to a maximum of a few hundred Watts.

It is the object of the present invention to introduce a new apparatus for repairing and regenerating cardiac tissues and for the electro-physiological, metabolic optimization of the heart, comprising a catheter adapted to be inserted, by means of a surgical incision or natural access, in appropriate position in the patient's heart, and a radiofrequency generator, associated with said catheter, adapted to emit a low-power radiofrequency electromagnetic field such as to not ingenerate local heating in the treated tissues.

### BRIEF DESCRIPTION OF THE DRAWINGS

Further features and advantages of the present invention will become more apparent from the following detailed description, provided by way of non-limitative example and shown in the accompanying figure, in which:

FIG. 1 shows a functional block chart of the apparatus according to the present invention.

### SUMMARY OF THE INVENTION

It is the object of the present invention to provide an apparatus comprising a catheter associated with a radiofrequency radio-electric signal generator which, by means of a surgical incision or natural access, is adapted to convey said radio-electric signal onto the cardiac surfaces, in order to repair, regenerate and normalize the electro-physiological, metabolic activities of the heart, even at conduction node and pathway level, in order to restore rhythms and lost functions, such as, for example, altered cardiac conduction and propagation speeds, cardiac rhythm disturbances (both atrial and ventricular), and to repair and regenerate cardiac distress areas, such as fibrotic and post-ischemic areas, and in general all the pathologies which results in alterations to the structure and function of the heart cells, such as cardiomyopathies.

### DETAILED DESCRIPTION OF THE INVENTION

The present description and the accompanying drawings are be understood as illustrative of and non-limiting the present invention, which may be implemented according to other, different embodiments, all within the scope of the appended claims, which also form an integral part of the text.

With reference to the accompanying figures, the present invention comprises an apparatus comprising a low invasive catheter and a low-power radiofrequency electromagnetic signal generator.

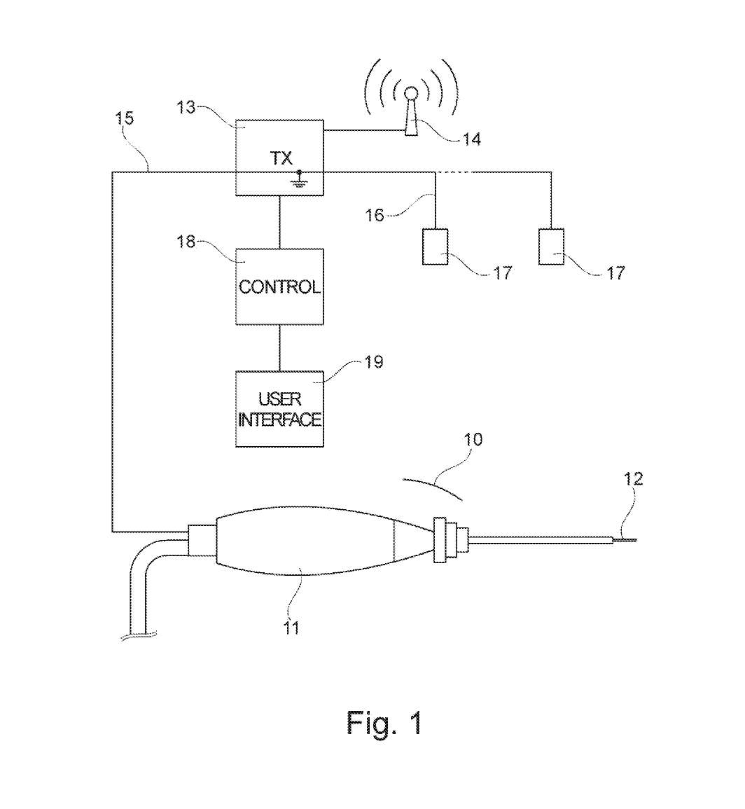

With reference to accompanying FIG. 1, said catheter 10 may be made in many manners adapted to the use according to the present invention. A preferred embodiment comprises an elongated body having a first end adapted to be grasped by the user, a second end adapted to be introduced into the patient's body, and an intermediate section, which divides said first end from said second end.

A control handle 11 is present at said first end of the body of catheter 10, while an electrode 12 is associated with said second end of catheter 10.

The body of said catheter 10 is preferably tubular and elongated having an axial central hole. Moreover, the body of catheter 10 is flexible, folding and substantially not compressible over its length, and may be made in any manner and of any suitable material. For example, said elongated body of catheter 10 may comprise an outer sheath made of materials such as polyurethane or elastomeric polymers adapted for surgical use, an inner reinforcing mesh made of stainless steel adapted to increase the torsional rigidity thereof so that when the control handle 11 is rotated, the intermediate segment of catheter 10 rotates correspondingly, and a possible outer stiffening tube to provide the elongated body of catheter 10 with higher torsional stability.

The size of said central axial hole is sufficient to accommodate, for example, a given number of elements chosen from the group comprising: a traction cable, at least one electric wire connected to said electrode, one or more tubes which allow to convey liquids or drugs for irrigating the part to be treated or to be injected into the part to be treated, means adapted to allow the lighting and/or viewing of the part to be treated.

Said catheter 10 may be of the telescopic type, i.e. it may comprise a main body provided with at least one telescopic part adapted to be extracted once it has been appropriately positioned at the part to be treated.

The apparatus according to the present invention further comprises a low-power radiofrequency generator. An example of said low-frequency radiofrequency generator is described in patent EP1301241 by the applicants of the present patent application, the contents of which is to be understood as incorporated by reference in the present patent application.

Said low-power radiofrequency generator comprises at least one transmitter 13 adapted to produce a radiofrequency electromagnetic field, where radiofrequency electromagnetic field means an electric signal or a high-frequency electromagnetic wave which propagates in air with an oscillation frequency which may be from a few kHz to 300 GHz and with correspondingly varying wavelengths from a few 100 km (at 3 kHz) to 1 mm (at 300 GHz).

Said transmitter 13 is associated with a control module 18 associated, in turn, with an appropriate user interface 19, at least one antenna 14 and at least one electric conductor 15 connected, at one end, to the ground reference of the circuitry of said transmitter 13 and, at the other end, to the electrode 12 of said catheter 10. Said transmitter 13 may be made according to the known methods of the prior art.

In a preferred embodiment of the present invention, said control module 19 further comprises programmable modulation means adapted to modulate the electromagnetic field produced by said transmitter 13. For example, said programmable modulation means may be of the ON/OFF type, substantially adapted to periodically enable and inhibit the emission of said electromagnetic field with ON and OFF times, which can be programmed by the user as desired.

In another preferred embodiment, said transmitter has an oscillation frequency of 2.45 GHz and a power, measured close to said antenna 14, lower than 100 mW.

The power of the radiofrequency electromagnetic field emitted during the operation of the apparatus according to the present invention is such not to cause noticeable heating effects on the patient's body.

During the patient's treatment, the apparatus according to the present invention is used as follows: the catheter 10, connected to the electric conductor 15 connected, in turn, to the ground reference of the circuitry of said transmitter 13, is appropriately introduced into the patient so as to reach the cardiac tissue to be treated. The transmitter is positioned so as to irradiate the patient's body, which is kept isolated from earth and from any other connection to ground, in a diffused manner by means of the generated electromagnetic field. Thereby, the patient's body will be concerned by a flow of radiofrequency currents having frequency, duration and modulation chosen by the medical operator according to a specific protocol. Said currents are thus conveyed, by the catheter 10 connected to the electric conductor 15 of said transmitter 13, so as to be concentrated at the part of the cardiac tissue subject to therapy.

The low-power radiofrequency generator of the apparatus according to the present invention is configured according to various operating protocols depending on the pathology or symptoms to be treated. For example, a single package of electromagnetic radiation lasting 350 ms and having an oscillation frequency of 2.45 GHz and a power, measured close to antenna 14, of about 10 mW, is such to determine positive variations of both activation and propagation of the cardiac electric activity.

A series of packages lasting 350 ms and spaced out by OFF periods lasting 2500 ms of electromagnetic radiation having an oscillation frequency of 2.45 GHz and a power, measured close to antenna 14, of about 10 mW is such to determine positive variations of both activation and propagation of the cardiac electric activity, and moreover is such to determine the repairing and/or regeneration of tissues.

The low-power radiofrequency generator of the apparatus according to the present invention may advantageously comprise a plurality of said electric conductors, wherein—in a preferred embodiment—a first conductor 15 is connected to an electrode 12 of said catheter 10 in the above-described modes, while a second conductor 16 is connected to at least one further electrode 17, which is applied, in appropriate position, on the patient's chest. In this embodiment, the apparatus according to the present invention is capable of boosting the endocardial treatment delivered by catheter 10 in order to stabilize and boost the described therapeutic effects.

In a further preferred embodiment of the present invention, the low-power radiofrequency generator of the apparatus according to the present invention may comprise a plurality of transmitters 13, each of which conveniently connected to a conductor 16 of said plurality of electric conductors, said conductor 16 being, in turn, conveniently connected to at least one further electrode 17. This allows to apply a plurality of electrodes to the patient, each concerned by radiofrequency currents with different frequency, modulation and intensity.

The use of the apparatus according to the present invention includes inserting catheter 10 into an appropriate blood vessel of the patient, then positioning the catheter in an intravascular position so as to place said electrode close to the part of cardiac tissue to be treated, stabilizing catheter 10 so as to maintain the reached position, positioning possible electrodes 17 on the patient, e.g. on the patient's chest, and irradiating the patient with the low-power electromagnetic field produced by said low-power radiofrequency generator.

The use of the apparatus and method according to the present invention allows to treat many pathological states of the cardiac muscle.

In cases of atrial fibrillation, in which extended hypovoltage areas are present in the cardiac muscle, the treatment by means of the apparatus and method according to the present invention allows to recover and restore in nearly complete manner said hypovoltage area by virtue of an optimization of the electro-physiological, metabolic features of the treated tissues.

In the cases of cardiac pathologies having altered conduction and propagation rates inside the electric conduction system of the heart, as in the cases of atrial fibrillation, the propagation region of the electric pulse in a reference time range appears considerably limited. After the treatment by means of the apparatus and method according to the present invention, said propagation region of the electric pulse, the reference time interval being equal, has a considerably wider surface by virtue of a substantial restoration of the electric functions of the treated cardiac tissues.

Finally, in the cases of myocardial infarction, the post-ischemic distress area may be considerably reduced as a result of the treatment by means of the apparatus and method according to the present invention.

1. An apparatus for repairing and regenerating cardiac tissues and for the electro-physiological, metabolic optimization of the heart, comprising:
a catheter, provided with an elongated body having a first end adapted to be grasped by the user and a second end adapted to be introduced into the patient's body;
a control handle, associated with said first end;
at least one electrode, associated with said second end;
a low-power radiofrequency generator, associated with said at least one electrode, comprising in turn
at least one transmitter, adapted to produce a radiofrequency electromagnetic field;
a control module, associated with said transmitter, and a user interface associated with said control module;
at least one antenna, associated with said transmitter;
at least one electric conductor connected, at one end, to the reference ground of the circuitry of said transmitter and, at the other end, to said at least one electrode.

2. The apparatus according to claim 1, wherein said catheter further comprises at least one element chosen from the group comprising: a traction cable, at least one electric wire connected to said electrode, one or more tubes which allow to convey liquids or drugs for irrigating the part to be treated or to be injected into the part to be treated, means adapted to allow the lighting and/or viewing of the part to be treated.

3. The apparatus according to claim 1, wherein said catheter comprises an outer sheath made of plastic material adapted for surgical use, an inner reinforcing mesh made of stainless steel adapted to increase the torsional rigidity thereof, and an outer stiffening tube to provide the body of said catheter with better torsional stability.

4. The apparatus according to claim 1, wherein said control module further comprises programmable modulation means adapted to modulate the electromagnetic field produced by said transmitter.

5. The apparatus according to claim 4, wherein said programmable modulating means are of the ON/OFF type, adapted to periodically enable and inhibit the emission of said electromagnetic field, with programmable ON times, during which said electromagnetic field is active, and programmable OFF times, during which said electromagnetic field is deactivated.

6. The apparatus according to claim 1, wherein said low-power radiofrequency generator comprises a plurality of said electric conductors, wherein a first conductor is connected to the electrode of said catheter and the other conductors are connected to further electrodes adapted to be externally applied to the patient.

7. The apparatus according to claim 1, wherein said transmitter has an oscillation frequency of 2.45 GHz and a power, measured close to said antenna, lower than 100 mW.

8. The apparatus according to claim 7, wherein said transmitter is adapted to emit an electromagnetic radiofrequency field having a frequency of about 2.45 GHz and a power, measured close to said antenna, of about 10 mW, and is modulated with ON times lasting about 350 ms.

9. The apparatus according to claim 8, wherein said electromagnetic radiofrequency field is modulated with OFF times lasting about 2500 ms.

10. A method for repairing and regenerating cardiac tissues and for the electro-physiological, metabolic optimization of the heart, comprising the following steps:
providing a catheter comprising a control handle associated with a first end of said catheter, at least one electrode associated with a second end of said catheter, a low-power radiofrequency generator associated with said at least one electrode, said radiofrequency generator comprising at least one transmitter adapted to produce an electromagnetic radiofrequency field, a control module associated with said transmitter, a user interface associated with said control module, at least one antenna associated with said transmitter, at least one electric conductor connected, at one end, to the ground reference of the circuitry of said transmitter and, at the other end, to said at least one electrode;
inserting said catheter into a patient's blood vessel;
positioning said catheter in an intravascular position close to the portion of cardiac tissue to be treated;
stabilizing said catheter;
irradiating the patient with the electromagnetic field produced by said radiofrequency generator.

11. The method according to claim 10, wherein said low-power radiofrequency generator comprises a plurality of said electric conductors, wherein a first conductor is connected to the electrode of said catheter and the other conductors are connected to further electrodes adapted to be externally applied to the patient.

12. The method according to claim 10, comprising the further following step of:
applying said further electrodes to the patient's chest.

13. The method according to claim 11, wherein said transmitter has an oscillation frequency of 2.45 GHz and a power, measured close to said antenna, lower than 100 mW.

14. The method according to claim 11, wherein said control module comprises further programmable modulation means adapted to modulate the electromagnetic field produced by said transmitter.

15. The method according to claim 12, wherein said transmitter is adapted to emit an electromagnetic radiofrequency field having a frequency of about 2.45 GHz and a power, measured close to said antenna, of about 10 mW, and is modulated with ON times lasting about 350 ms.
